(19) United States
(12) Patent Application Publication
(10) Pub. No.: US 2025/0025237 A1
(43) Pub. Date: Jan. 23, 2025

(54) SYSTEM AND METHOD FOR PRESSURE MONITORING WITHIN A CATHETER SYSTEM

(71) Applicant: BOLT MEDICAL, INC., Carlsbad, CA (US)

(72) Inventors: Christopher A. Cook, Laguna Niguel, CA (US); Eric Schultheis, San Clemente, CA (US)

(21) Appl. No.: 18/904,579

(22) Filed: Oct. 2, 2024

Related U.S. Application Data
(63) Continuation-in-part of application No. 17/154,453, filed on Jan. 21, 2021.
(60) Provisional application No. 62/985,452, filed on Mar. 5, 2020.
(60) Provisional application No. 62/972,268, filed on Feb. 10, 2020.

(51) Int. Cl. A61B 18/26; A61B 18/00; A61B 18/22

(52) U.S. Cl. CPC A61B 18/26; A61B 2018/00404; A61B 2018/00773; A61B 2018/2253; A61B 2018/263; A61B 2018/266

(57) ABSTRACT

A catheter system (100) for treating a treatment site (106) includes an energy source (124), a balloon (104), an energy guide (122A), an inflation conduit (140) and a pressure sensor assembly (142). The balloon (104) is positionable substantially adjacent to the treatment site (106). The balloon (104) has a balloon wall (130) that defines a balloon interior (146) that receives a balloon fluid (132). The energy source (124) generates energy that is received by the energy guide (122A) so that the energy guide (122A) can guide the light energy into the balloon interior (146). The inflation conduit (140) is in fluid communication with the balloon interior (146). The inflation conduit (140) is configured to convey the balloon fluid (132) into the balloon interior (146). The pressure sensor assembly (142) is configured to sense a balloon pressure of the balloon fluid (132) within the balloon interior (146). The pressure sensor assembly (142) is in fluid communication with the balloon interior (146) and the inflation conduit (140).

### RELATED APPLICATIONS

The present application is a continuation-in-part application claiming the benefit of priority under 35 U.S.C. 120 on U.S. patent application Ser. No. 17/154,453, filed on Jan. 21, 2021. Additionally, U.S. patent application Ser. No. 17/154,453 claims priority on U.S. Provisional Application Ser. No. 62/972,268, filed on Feb. 10, 2020, entitled “SYSTEM AND METHOD FOR PRESSURE MONITORING WITHIN LITHOPLASTY DEVICE,” and on U.S. Provisional Application Ser. No. 62/985,452, filed on Mar. 5, 2020, entitled “SYSTEM AND METHOD FOR PRESSURE MONITORING WITHIN LITHOPLASTY DEVICE.” As far as permitted, the contents of U.S. patent application Ser. No. 17/154,453 and U.S. Provisional Application Ser. Nos. 62/972,268 and 62/985,452 are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

### SUMMARY

The present invention is directed toward a catheter system for placement within a blood vessel having a vessel wall. The catheter system can be used for treating a treatment site within or adjacent to the vessel wall. In various embodiments, the catheter system includes an energy source, a balloon, an energy guide, an inflation conduit, and a pressure sensor assembly. The energy source generates energy. The balloon is positionable substantially adjacent to the treatment site. The balloon has a balloon wall that defines a balloon interior. The balloon interior is configured to receive a balloon fluid. The energy guide is configured to receive energy from the energy source. The energy guide guides the energy into the balloon interior. The inflation conduit is in fluid communication with the balloon interior. The inflation conduit is configured to convey the balloon fluid into the balloon interior. The pressure sensor assembly is configured to sense a balloon pressure of the balloon fluid within the balloon interior. The pressure sensor assembly is in fluid communication with the balloon interior and the inflation conduit.

In some embodiments, the catheter system further comprises a handle assembly that is coupled to the balloon, the handle assembly being positioned spaced apart from the balloon, the handle assembly being usable by a user to operate the catheter system, the pressure sensor assembly being positioned within the handle assembly.

In various embodiments, the catheter system further comprises a manifold assembly that is coupled to the handle assembly, the manifold assembly being in fluid communication with (i) the balloon fluid within the balloon interior, (ii) the pressure sensor assembly, and (iii) the inflation conduit.

In certain embodiments, the inflation fluid is configured to flow first through the inflation conduit and subsequently through the manifold assembly before entering the balloon interior.

In some embodiments, the manifold assembly includes a manifold retainer that is configured to receive and retain the balloon fluid.

In various embodiments, the pressure sensor assembly includes a pressure sensor that contacts the manifold retainer so that the pressure sensor can sense the balloon pressure of the balloon fluid based on the balloon fluid retained within the manifold retainer.

In certain embodiments, the pressure sensor assembly includes a pressure sensor, the pressure sensor including a printed circuit board.

In some embodiments, the manifold assembly includes a manifold body, the pressure sensory assembly being coupled to the manifold body so that the pressure sensor extends within the manifold body.

In various embodiments, the pressure sensor assembly includes a pressure sensor sealer that forms a seal between the pressure sensor and the manifold body.

In certain embodiments, the manifold body includes a pressure sensor aperture that is configured to receive the pressure sensor.

The present invention is also directed toward a catheter system for placement within a blood vessel having a vessel wall. The catheter system can be used for treating a treatment site within or adjacent to the vessel wall. In various embodiments, the catheter system includes a balloon, an inflation conduit, and a pressure sensor assembly. The balloon is positionable substantially adjacent to the treatment site. The balloon has a balloon wall that defines a balloon interior. The balloon interior is configured to receive a balloon fluid. The inflation conduit is in fluid communication with the balloon interior. The inflation conduit is configured to convey the balloon fluid into the balloon interior. The pressure sensor assembly is configured to sense a balloon pressure of the balloon fluid within the balloon interior. The pressure sensor assembly is in fluid communication with the balloon interior and the inflation conduit.

In some embodiments, the catheter system further comprises a handle assembly that is coupled to the balloon, the handle assembly being positioned spaced apart from the balloon, the handle assembly being usable by a user to operate the catheter system, the pressure sensor assembly being positioned within the handle assembly.

In various embodiments, the catheter system further comprises a manifold assembly that is coupled to the handle assembly, the manifold assembly being in fluid communication with (i) the balloon fluid within the balloon interior, (ii) the pressure sensor assembly, and (iii) the inflation conduit.

In certain embodiments, the inflation fluid is configured to flow first through the inflation conduit and subsequently through the manifold assembly before entering the balloon interior.

In some embodiments, the manifold assembly includes a manifold retainer that is configured to receive and retain the balloon fluid.

In various embodiments, the pressure sensor assembly includes a pressure sensor that contacts the manifold retainer so that the pressure sensor can sense the balloon pressure of the balloon fluid based on the balloon fluid retained within the manifold retainer.

In certain embodiments, the pressure sensor assembly includes a pressure sensor, the pressure sensor including a printed circuit board.

In some embodiments, the manifold assembly includes a manifold body, the pressure sensory assembly being coupled to the manifold body so that the pressure sensor extends within the manifold body.

In various embodiments, the pressure sensor assembly includes a pressure sensor sealer that forms a seal between the pressure sensor and the manifold body.

In certain embodiments, the present invention is further directed toward a catheter system for placement within a blood vessel having a vessel wall. The catheter system can be used for treating a treatment site within or adjacent to the vessel wall. In various embodiments, the catheter system includes an energy source, a balloon, an energy guide, a handle assembly, an inflation conduit, and a pressure sensor assembly. The energy source generates energy. The balloon is positionable substantially adjacent to the treatment site. The balloon has a balloon wall that defines a balloon interior. The balloon interior is configured to receive a balloon fluid. The energy guide is configured to receive energy from the energy source. The energy guide guides the energy into the balloon interior. The handle assembly is coupled to the balloon. The handle assembly is positioned spaced apart from the balloon. The handle assembly is usable by a user to operate the catheter system. The inflation conduit is in fluid communication with the balloon interior. The inflation conduit is coupled to the handle assembly. The inflation conduit is configured to convey the balloon fluid into the balloon interior. The pressure sensor assembly is configured to sense a balloon pressure of the balloon fluid within the balloon interior. The pressure sensor assembly is in fluid communication with the balloon interior and the inflation conduit. The pressure sensor assembly is positioned within the handle assembly.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X.sub.1-X.sub.n, Y.sub.1-Y.sub.m, and Z.sub.1-Z.sub.o, the phrase is intended to refer to a single element selected from X, Y, and Z, a combination of elements selected from the same class (for example, X.sub.1 and X.sub.2) as well as a combination of elements selected from two or more classes (for example, Y.sub.1 and Z.sub.o).

It is to be noted that the term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of an embodiment of a catheter system in accordance with various embodiments herein, the catheter system including a pressure sensor assembly;

FIG. 2 is a schematic cross-sectional view of a portion of an embodiment of the catheter system, including an embodiment of the pressure sensor assembly;

FIG. 3 is a schematic cross-sectional view of a portion of an embodiment of the catheter system, including another embodiment of the pressure sensor assembly;

FIG. 4A is a schematic cross-sectional view of a portion of an embodiment of the catheter system, including another embodiment of the pressure sensor assembly;

FIG. 4B is a partially exploded side view of a portion of an embodiment of the catheter system, including another embodiment of the pressure sensor assembly and a manifold assembly;

FIG. 4C is a cross-sectional view of a portion of the embodiment of the catheter system taken along line 4C-4C in FIG. 4B;

FIG. 4D is a top view of a portion of the embodiment of the catheter system illustrated in FIGS. 4A-4C; and

FIG. 4E is a cross-sectional view of a portion of the embodiment of the catheter system taken along line 4E-4E in FIG. 4D.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

The catheter systems and related methods disclosed herein are configured to monitor the performance, reliability and safety of a catheter. In various embodiments, the catheter systems of the present invention utilize an energy source, e.g., a light source such as a laser source in certain embodiments or another suitable energy source, which provides energy that is guided by an energy guide to create a localized plasma in a balloon fluid within a balloon interior of an inflatable balloon of the catheter. This localized plasma, in turn, induces a high energy bubble inside the balloon interior to create pressure waves and/or pressure waves to impart pressure onto and induce fractures in a treatment site, such as a calcified vascular lesion or a fibrous vascular lesion, at the treatment site within or adjacent to a blood vessel wall. As used herein, the treatment site can include a vascular lesion, such as a calcified vascular lesion or a fibrous vascular lesion, typically found in a blood vessel and/or at or near a heart valve, such as the mitral valve or the aortic valve, as non-exclusive examples.

Importantly, as described in detail herein, the catheter systems of the present invention include a pressure sensor that is configured to monitor the pressure of a balloon fluid that is retained within the balloon interior of the balloon. Specific examples of issues that are addressed by the present invention include, but are not limited to: (1) detection of rupturing or bursting of the balloon, (2) detection of the successful firing of the plasma generators, i.e., the energy source, (3) detection of failure of the plasma generators, and (4) monitoring of progression of the procedure and efficacy of treatment.

In particular, in various embodiments, the catheter systems can include a catheter configured to advance to the treatment site within or adjacent a blood vessel or heart valve. The catheter includes a catheter shaft, and a balloon that is coupled and/or secured to the catheter shaft. The balloons herein can include a balloon wall that defines a balloon interior and can be configured to receive a balloon fluid within the balloon interior to expand from a collapsed configuration suitable for advancing the catheter through a patient's vasculature, to an expanded configuration suitable for anchoring the catheter in position relative to the treatment site. The catheter systems also include one or more energy guides, e.g., light guides in certain embodiments, disposed along the catheter shaft and within the balloon. Each energy guide can be configured for generating pressure waves within the balloon for disrupting the vascular lesions. The catheter systems utilize energy from an energy source, e.g., light energy from a light source in certain embodiments, to generate a plasma within the balloon fluid at or near a guide distal end of the energy guide disposed in the balloon located at the treatment site. The plasma formation can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles can generate one or more pressure waves within the balloon fluid retained within the balloon and thereby impart pressure waves upon the treatment site. In some embodiments, the energy source can be configured to provide sub-millisecond pulses of energy from the energy source to initiate plasma formation in the balloon fluid within the balloon to cause rapid bubble formation and to impart pressure waves upon the balloon wall at the treatment site. Thus, the pressure waves can transfer mechanical energy through an incompressible balloon fluid to the treatment site to impart a fracture force on the vascular lesion.

Additionally, the catheter systems further include the pressure sensor that can be positioned at any appropriate position within the catheter system. As described in detail herein, the pressure sensor is configured to sense and/or monitor a fluid pressure (also sometimes referred to herein as a “balloon pressure”) of the balloon fluid within the balloon interior during operation of the catheter system. The pressure sensor can generate sensor output relevant to the sensed fluid pressure and provide such sensor output to a system controller that is configured to control various operations of the catheter system. This sensing and/or monitoring of the fluid pressure with the pressure sensor provides valuable information to the user or operator as to the performance, reliability and safety of the catheter system.

As used herein, the terms “intravascular lesion,” “vascular lesion,” and/or “treatment site” are used interchangeably unless otherwise noted. As such, the intravascular lesions and/or the vascular lesions are sometimes referred to herein simply as “lesions.”

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

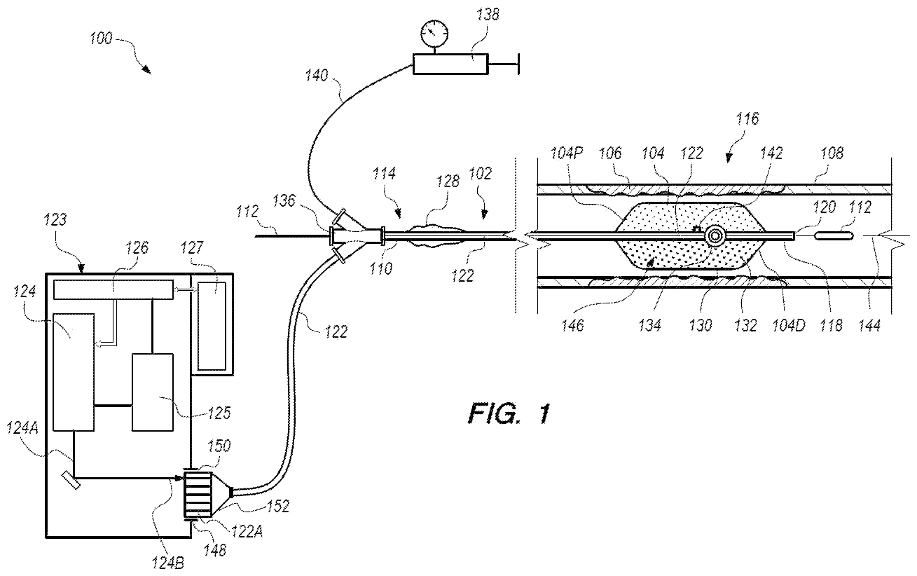

It is appreciated that the catheter systems disclosed herein can include many different forms. Referring now to FIG. 1, a schematic cross-sectional view is shown of a catheter system 100 in accordance with various embodiments herein. As described herein, the catheter system 100 is suitable for imparting pressure to induce fractures in one or more treatment sites 106 within or adjacent a vessel wall of a blood vessel. In the embodiment illustrated in FIG. 1, the catheter system 100 can include one or more of a catheter 102, an energy guide bundle 122 (also sometimes referred to herein as a “light guide bundle”) including one or more energy guides 122A (sometimes referred to herein as “light guides”), a source manifold 136, a fluid pump 138, a system console 123 including one or more of a light source 124 (sometimes referred to herein as an “energy source”), a power source 125, a system controller 126, and a graphic user interface 127 (a “GUI”), a handle assembly 128, and a pressure sensor assembly 142.

The catheter 102 is configured to move to a treatment site 106 within or adjacent to a blood vessel 108. The treatment site 106 can include one or more vascular lesions, such as calcified vascular lesions, for example. Additionally, or in the alternative, the treatment site 106 can include vascular lesions such as fibrous vascular lesions.

The catheter 102 can include an inflatable balloon 104 (sometimes referred to herein simply as a “balloon”), a catheter shaft 110 and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter shaft 110 can also include a guidewire lumen 118, which is configured to move over the guidewire 112. The catheter shaft 110 can further include an inflation lumen (not shown). In some embodiments, the catheter 102 can have a distal end opening 120 and can accommodate and be tracked over the guidewire 112 as the catheter 102 is moved and positioned at or near the treatment site 106.

In various embodiments, the catheter shaft 110 of the catheter 102 can be coupled to the one or more light guides 122A of the light guide bundle 122 that are in optical communication with the light source 124. The light guide(s) 122A can be disposed along the catheter shaft 110 and within the balloon 104. In some embodiments, each light guide 122A can be an optical fiber, and the light source 124 can be a laser. The light source 124 can be in optical communication with the light guides 122A at the proximal portion 114 of the catheter system 100.

In some embodiments, the catheter shaft 110 can be coupled to multiple light guides 122A, such as a first light guide, a second light guide, a third light guide, etc., which can be disposed at any suitable position about the guidewire lumen 118 and/or the catheter shaft 110. For example, in certain non-exclusive embodiments, two light guides 122A can be spaced apart by approximately 180 degrees about the circumference of the guidewire lumen 118 and/or the catheter shaft 110; three light guides 122A can be spaced apart by approximately 120 degrees about the circumference of the guidewire lumen 118 and/or the catheter shaft 110; or four light guides 122A can be spaced apart by approximately 90 degrees about the circumference of the guidewire lumen 118 and/or the catheter shaft 110. Still alternatively, multiple light guides 122A need not be uniformly spaced apart from one another about the circumference of the guidewire lumen 118 and/or the catheter shaft 110. More particularly, it is further appreciated that the light guides 122A described herein can be disposed uniformly or non-uniformly about the guidewire lumen 118 and/or the catheter shaft 110 to achieve the desired effect in the desired locations.

The balloon 104 can include a balloon wall 130 that defines a balloon interior 146, and can be inflated with a balloon fluid 132 to expand from a collapsed configuration suitable for advancing the catheter 102 through a patient's vasculature, to an expanded configuration suitable for anchoring the catheter 102 in position relative to the treatment site 106. Stated in another manner, when the balloon 104 is in the expanded configuration, the balloon wall 130 of the balloon 104 is configured to be positioned substantially adjacent to the treatment site 106. In some embodiments, the light source 124 of the catheter system 100 can be configured to provide sub-millisecond pulses of light from the light source 124, along the light guides 122A, to a location within the balloon interior 146 of the balloon 104, thereby inducing plasma formation in the balloon fluid 132 within the balloon interior 146 of the balloon 104. The plasma formation causes rapid bubble formation, and imparts pressure waves upon the treatment site 106. Exemplary plasma-induced bubbles are shown as bubbles 134 in FIG. 1.

It is appreciated that although the catheter systems 100 illustrated herein are generally described as including a light source 124 and one or more light guides 122A, the catheter system 100 can alternatively and equally include and/or utilize any suitable energy source and energy guides for purposes of generating the desired plasma in the balloon fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, forms the pressure waves within the balloon fluid 132 that are utilized to provide the fracture force onto the treatment site 106. Still, alternatively, the energy source 124 and/or the energy guides 122A can have another suitable design. Thus, although certain embodiments shown and described herein focus on “light sources” and “light guides,” etc., it is understood that this is not intended to limit the disclosure herein to optical systems. Rather, it is recognized that other types of energy sources and energy guides can equally be utilized with the systems and methods provided herein, and that “light sources” and “light guides,” etc., are provided by way of example and not by way of limitation, for ease in understanding the present disclosure.

The balloons 104 suitable for use in the catheter systems 100 described in detail herein include those that can be passed through the vasculature of a patient when in the collapsed configuration. In some embodiments, the balloons 104 herein are made from silicone. In other embodiments, the balloons 104 herein are made from polydimethylsiloxane (PDMS), polyurethane, polymers such as PEBAX™ material available from Arkema, which has a location at King of Prussia, Pennsylvania, USA, nylon, and the like. In some embodiments, the balloons 104 can include those having diameters ranging from one millimeter (mm) to 25 mm in diameter. In some embodiments, the balloons 104 can include those having diameters ranging from at least 1.5 mm to 12 mm in diameter. In some embodiments, the balloons 104 can include those having diameters ranging from at least one mm to five mm in diameter.

In some embodiments, the balloons 104 herein can include those having a length ranging from at least five mm to 300 mm. More particularly, in some embodiments, the balloons 104 herein can include those having a length ranging from at least eight mm to 200 mm. It is appreciated that balloons 104 of greater length can be positioned adjacent to larger treatment sites 106, and, thus, may be usable for imparting pressure onto and inducing fractures in larger treatment sites 106 or multiple treatment sites 106.

The balloons 104 herein can be inflated to inflation pressures (also referred to as “fluid pressure” and/or “balloon pressure”) of between approximately one atmosphere (atm) to 70 atm. In some embodiments, the balloons 104 herein can be inflated to inflation pressures of from at least 20 atm to 70 atm. In other embodiments, the balloons 104 herein can be inflated to inflation pressures of from at least six atm to 20 atm. In still other embodiments, the balloons 104 herein can be inflated to inflation pressures of from at least three atm to 20 atm. In yet other embodiments, the balloons 104 herein can be inflated to inflation pressures of from at least two atm to ten atm.

The balloons 104 herein can include those having various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloons 104 herein can include a drug-eluting coating or a drug-eluting stent structure. The drug-eluting coating or drug-eluting stent can include one or more therapeutic agents, including anti-inflammatory agents, anti-neoplastic agents, anti-angiogenic agents, and the like.

The balloon fluid 132 can be a liquid or a gas. Exemplary balloon fluids 132 suitable for use herein can include, but are not limited to, one or more of water, saline, contrast medium, fluorocarbons, perfluorocarbons, gases, such as carbon dioxide, and the like. In some embodiments, the balloon fluids 132 described can be used as base balloon fluids. In some embodiments, the balloon fluids 132 include a mixture of saline to contrast medium in a volume ratio of 50:50. In other embodiments, the balloon fluids 132 include a mixture of saline to contrast medium in a volume ratio of 25:75. In still other embodiments, the balloon fluids 132 include a mixture of saline to contrast medium in a volume ratio of 75:25. Additionally, the balloon fluids 132 suitable for use herein can be tailored on the basis of composition, viscosity, and the like in order to manipulate the rate of travel of the pressure waves therein. In certain embodiments, the balloon fluids 132 suitable for use herein are biocompatible. A volume of balloon fluid 132 can be tailored by the chosen light source 124 and the type of balloon fluid 132 used.

In some embodiments, the contrast agents used in the contrast media herein can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine-containing contrast agents can include gadolinium (Ill)-based contrast agents. Suitable fluorocarbon and perfluorocarbon agents can include, but are not to be limited to, agents such as the perfluorocarbon dodecafluoropentane (DDFP, C5F12).

The balloon fluids 132 herein can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the balloon fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system. By way of non-limiting examples, various lasers described herein can include neodymium:yttrium-aluminum-garnet (Nd:YAG—emission maximum=1064 nm) lasers, holmium:YAG (Ho:YAG—emission maximum=2.1 μm) lasers, or erbium:YAG (Er:YAG—emission maximum=2.94 μm). In some embodiments, the absorptive agents used herein can be water-soluble. In other embodiments, the absorptive agents used herein are not water-soluble. In some embodiments, the absorptive agents used in the balloon fluids 132 herein can be tailored to match the peak emission of the light source 124. Various light sources 124 having emission wavelengths of at least ten nanometers to one millimeter are discussed elsewhere herein.

It is appreciated that the catheter system 100 and/or the light guide bundle 122 disclosed herein can include any number of light guides 122A in optical communication with the light source 124 at the proximal portion 114, and with the balloon fluid 132 within the balloon interior 146 of the balloon 104 at the distal portion 116. For example, in some embodiments, the catheter system 100 and/or the light guide bundle 122 can include from one light guide 122A to five light guides 122A. In other embodiments, the catheter system 100 and/or the light guide bundle 122 can include from five light guides 122A to fifteen light guides 122A. In yet other embodiments, the catheter system 100 and/or the light guide bundle 122 can include from ten light guides 122A to thirty light guides 122A. Alternatively, in still other embodiments, the catheter system 100 and/or the light guide bundle 122 can include greater than 30 light guides 122A.

It is further appreciated that the light guides 122A can be disposed at any suitable position about the circumference of the guidewire lumen 118 and/or the catheter shaft 110, and the guide distal end of each of the light guides 122A can be disposed at any suitable longitudinal position relative to the length of the balloon 104 and/or relative to the length of the guidewire lumen 118.

The light guides 122A herein can assume many configurations about and/or relative to the catheter shaft 110 of the catheters 102 described herein. In some embodiments, the light guides 122A can run parallel to the longitudinal axis 144 of the catheter shaft 110. In some embodiments, the light guides 122A can be physically coupled to the catheter shaft 110. In other embodiments, the light guides 122A can be disposed along a length of an outer diameter of the catheter shaft 110. In yet other embodiments, the light guides 122A herein can be disposed within one or more light guide lumens within the catheter shaft 110.

The source manifold 136 can be positioned at or near the proximal portion 114 of the catheter system 100. The source manifold 136 can include one or more proximal end openings that can receive the plurality of light guides 122A of the light guide bundle 122, the guidewire 112, and/or an inflation conduit 140 that is coupled in fluid communication with the fluid pump 138. The catheter system 100 can also include the fluid pump 138 that is configured to inflate the balloon 104 with the balloon fluid 132 as needed.

As noted above, in the embodiment illustrated in FIG. 1, the system console 123 can include one or more of: the light source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still, alternatively, one or more of the light source 124, the power source 125, the system controller 126, and the GUI 127 can be provided within the catheter system 100 without the specific need for the system console 123.

The system console 123, and the components included therewith, is operatively coupled to the catheter 102, the light guide bundle 122, and the remainder of the catheter system 100. For example, in some embodiments, as illustrated in FIG. 1, the system console 123 can include a console connection aperture 148 (also sometimes referred to generally as a “socket”) by which the light guide bundle 122 is mechanically coupled to the system console 123. In such embodiments, the light guide bundle 122 can include a guide coupling housing 150 (also sometimes referred to generally as a “ferrule”) that houses a portion, e.g., a guide proximal end, of each of the light guides 122A. The guide coupling housing 150 is configured to fit and be selectively retained within the console connection aperture 148 to provide the desired mechanical coupling between the light guide bundle 122 and the system console 123.

The light guide bundle 122 can also include a guide bundler 152 (or “shell”) that brings each of the individual light guides 122A closer together so that the light guides 122A and/or the light guide bundle 122 can be in a more compact form as it extends with the catheter 102 into the blood vessel 108 during use of the catheter system 100.

As provided herein, the light source 124 can be selectively and/or alternatively coupled in optical communication with each of the light guides 122A in the light guide bundle 122. In particular, the light source 124 is configured to generate light energy in the form of a source beam 124A, e.g., a pulsed source beam, that can be selectively and/or alternatively directed to and received by each of the light guides 122A in the light guide bundle 122 as an individual guide beam 124B. Alternatively, the catheter system 100 can include more than one light source 124. For example, in one non-exclusive alternative embodiment, the catheter system 100 can include a separate light source 124 for each of the light guides 122A in the light guide bundle 122.

The light source 124 can have any suitable design. In certain embodiments, as noted above, the light source 124 can be configured to provide sub-millisecond pulses of light from the light source 124, that are directed along the light guides 122A, to a location within the balloon 104, thereby inducing plasma formation in the balloon fluid 132 within the balloon 104. The plasma formation causes rapid bubble formation, and imparts pressure waves upon the treatment site 106. In such embodiments, the sub-millisecond pulses of light from the light source 124 can be delivered to the treatment site 106 at a frequency of between approximately one hertz (Hz) and 5000 Hz. In some embodiments, the sub-millisecond pulses of light from the light source 124 can be delivered to the treatment site 106 at a frequency of between approximately 30 Hz and 1000 Hz. In other embodiments, the sub-millisecond pulses of light from the light source 124 can be delivered to the treatment site 106 at a frequency of between approximately ten Hz and 100 Hz. In yet other embodiments, the sub-millisecond pulses of light from the light source 124 can be delivered to the treatment site 106 at a frequency of between approximately one Hz and 30 Hz. Alternatively, the sub-millisecond pulses of light can be delivered to the treatment site 106 at a frequency that can be greater than 5000 Hz.

It is appreciated that although the light source 124 is typically utilized to provide pulses of light energy, the light source 124 can still be described as providing a single source beam 124A, i.e., a single pulsed source beam.

The light sources 124 suitable for use herein can include various types of light sources, including lasers and lamps. Alternatively, as noted above, the light sources 124, as referred to herein, can include any suitable type of energy source, such as a high voltage energy source that provides high voltage pulses of energy.

Suitable lasers can include short pulse lasers on the sub-millisecond timescale. In some embodiments, the light source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the balloon fluid 132 of the catheters 102 described herein. In various embodiments, the pulse widths can include those falling within a range, including from at least ten ns to 200 ns. In some embodiments, the pulse widths can include those falling within a range, including from at least 20 ns to 100 ns. In other embodiments, the pulse widths can include those falling within a range, including from at least one ns to 500 ns.

Exemplary nanosecond lasers can include those within the UV to IR spectrum, spanning wavelengths of about ten nanometers (nm) to one millimeter (mm). In some embodiments, the light sources 124 suitable for use in the catheter systems 100 herein can include those capable of producing light at wavelengths of from at least 750 nm to 2000 nm. In other embodiments, the light sources 124 can include those capable of producing light at wavelengths of from at least 700 nm to 3000 nm. In still other embodiments, the light sources 124 can include those capable of producing light at wavelengths of from at least 100 nm to ten micrometers (μm). Nanosecond lasers can include those having repetition rates of up to 200 kHz. In some embodiments, the laser can include a Q-switched thulium:yttrium-aluminum-garnet (Tm:YAG) laser. In other embodiments, the laser can include a neodymium:yttrium-aluminum-garnet (Nd:YAG), holmium:yttrium-aluminum-garnet (Ho:YAG), erbium:yttrium-aluminum-garnet (Er:YAG), excimer laser, helium-neon laser, carbon dioxide laser, as well as doped, pulsed, fiber lasers.

The catheter systems 100 disclosed herein can generate pressure waves having maximum pressures in the range of at least one megapascal (MPa) to 100 MPa. The maximum pressure generated by a particular catheter system 100 will depend on the light source 124, the absorbing material, the bubble expansion, the propagation medium, the balloon material, and other factors. In some embodiments, the catheter systems 100 herein can generate pressure waves having maximum pressures in the range of at least two MPa to 50 MPa. In other embodiments, the catheter systems 100 herein can generate pressure waves having maximum pressures in the range of at least two MPa to 30 MPa. In yet other embodiments, the catheter systems 100 herein can generate pressure waves having maximum pressures in the range of at least 15 MPa to 25 MPa.

The pressure waves described herein can be imparted upon the treatment site 106 from a distance within a range from at least 0.1 millimeters (mm) to 25 mm extending radially from the light guides 122A when the catheter 102 is placed at the treatment site 106. In some embodiments, the pressure waves can be imparted upon the treatment site 106 from a distance within a range from at least ten mm to 20 mm extending radially from the light guides 122A when the catheter 102 is placed at the treatment site 106. In other embodiments, the pressure waves can be imparted upon the treatment site 106 from a distance within a range from at least one mm to ten mm extending radially from the light guides 122A when the catheter 102 is placed at the treatment site 106. In yet other embodiments, the pressure waves can be imparted upon the treatment site 106 from a distance within a range from at least 1.5 mm to four mm extending radially from the light guides 122A when the catheter 102 is placed at the treatment site 106. In some embodiments, the pressure waves can be imparted upon the treatment site 106 from a range of at least two MPa to 30 MPa at a distance from 0.1 mm to ten mm. In some embodiments, the pressure waves can be imparted upon the treatment site 106 from a range of at least two MPa to 25 MPa at a distance from 0.1 mm to ten mm.

The power source 125 is electrically coupled to and is configured to provide necessary power to each of the light source 124, the system controller 126, the GUI 127, the handle assembly 128, and the pressure sensor assembly 142. The power source 125 can have any suitable design for such purposes.

The system controller 126 is electrically coupled to and receives power from the power source 125. Additionally, the system controller 126 is coupled to and is configured to control operation of each of the light source 124, the GUI 127 and the pressure sensor assembly 142. The system controller 126 can include one or more processors or circuits for purposes of controlling the operation of at least the light source 124, the GUI 127 and the pressure sensor assembly 142. For example, the system controller 126 can control the energy source 124, e.g., the light source, for generating pulses of energy, e.g., light energy, as desired, e.g., at any desired firing rate. Additionally, the system controller 126 can control the pressure sensor assembly 142 to effectively sense and/or monitor the fluid pressure (or balloon pressure) of the balloon fluid 132 within the balloon interior 146 of the balloon, i.e., to more effectively monitor the performance, reliability and safety of the catheter 102 and the catheter system 100. Further, in certain embodiments, the system controller 126 is configured to receive, process and integrate sensor output from the pressure sensor assembly 142 to determine and/or adjust for proper functioning of the catheter system 100. Stated in another manner, based at least in part on the sensor output from the pressure sensor assembly 142, the system controller 126 can determine that certain modifications to the functioning of the catheter system 100 are required. Further, the system controller 126 can also be configured to detect, determine or otherwise recognize various situations. For example, the system controller 126 can also be configured to detect, determine or otherwise recognize evidence of balloon rupture or light source malfunction or failure, and/or when the catheter system 100 is operating appropriately and effectively. The system controller 126 can then provide appropriate signals to the user via the GUI 137 in such certain situations, e.g., when the pressure sensor assembly 142 provides evidence of balloon rupture or light source malfunction or failure, and/or when the catheter system 100 is operating appropriately and effectively, or any other relatively important situation or status of the catheter system 100. Moreover, in some embodiments, the system controller 126 can be configured to automatically stop operation of the catheter system 100 when the sensor output dictates that such action would be appropriate.

The system controller 126 can further be configured to control operation of other components of the catheter system 100, e.g., the positioning of the catheter 102 adjacent to the treatment site 106, the inflation of the balloon 104 with the balloon fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. Additionally, the GUI 127 is electrically connected to the system controller 126. With such design, the GUI 127 can be used by the user or operator to ensure that the catheter system 100 is employed as desired to impart pressure onto and induce fractures into the treatment site 106. Additionally, the GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time, e.g., during use of the catheter system 100. Further, in various embodiments, the GUI 127 can include one or more colors, different sizes, varying brightness, etc., that may act as alerts to the user or operator. Additionally, or in the alternative, the GUI 127 can provide audio data or information to the user or operator. It is appreciated that the specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. Additionally, in this embodiment, the handle assembly 128 is coupled to the balloon 104 and is positioned spaced apart from the balloon 104. Alternatively, the handle assembly 128 can be positioned at another suitable location.

The handle assembly 128 is handled and used by the user or operator to operate, position and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the light source 124, the fluid pump 138, the GUI 127 and the pressure sensor assembly 142. In some embodiments, the handle assembly 128 can integrate and/or include at least a portion of the system controller 126 within an interior of the handle assembly 128. It is understood that the handle assembly 128 can include fewer or additional components than those specifically illustrated and described herein.

As an overview, and as provided in greater detail herein, the pressure sensor assembly 142 can sense and/or monitor a balloon pressure within the balloon interior 146 of the balloon 104. More specifically, the pressure sensor assembly 142 can provide real-time continuous monitoring of the balloon pressure within the balloon interior 146 in conjunction with electronics or optics that can be included in the handle assembly 128 and/or the system console 123. Further, the pressure sensor assembly 142 can generate a sensor signal or sensor output based at least in part on the balloon pressure, which is provided to other structures within the catheter system 100, e.g., the system controller 126. The system controller 126 can process and condition the sensor signal from the pressure sensor assembly 142 to look at changes in different time regimes. The system controller 126 can subsequently control various functions of the catheter system 100 as described herein based at least in part on the sensor signal or sensor output, i.e., based at least in part on the balloon pressure within the balloon interior 146 of the balloon 104.

As described in detail herein, the pressure sensor assembly 142 can be positioned in any suitable locations within the catheter system 100 so that it is in communication with the balloon fluid 132 within the balloon interior 146. For example, in certain embodiments, at least a portion of the pressure sensor assembly 142 can be positioned substantially within the balloon interior 146 of the balloon 104. Additionally, or in the alternative, at least a portion of the pressure sensor assembly 142 can be positioned substantially within the handle assembly 128. Further, or in the alternative, at least a portion of the pressure sensor assembly 125 can be positioned substantially between the handle assembly 128 and the balloon interior 146 of the balloon 104. Still further, or in the alternative, at least a portion of the pressure sensor assembly 142 can be positioned in another suitable location within the catheter system 100.

As noted above, the pressure sensor assembly 142 of the present invention addresses several important challenges with the performance, reliability and safety of a catheter, in particular one that utilizes an energy source, e.g., a light source 124, to create a localized plasma which in turn induces a high energy bubble inside the balloon 104. For example, as noted above, issues that are addressed by the present invention include, but are not limited to: (1) detection of rupturing or bursting of the balloon, (2) detection of successful firing of the plasma generators, i.e., the light source or other suitable energy source, (3) detection of failure of the plasma generators, i.e., the light source or other suitable energy source, and (4) monitoring of progression of the procedure and efficacy of treatment.

In particular, during use of the catheter system 100, the pressure sensor assembly 142 is configured to continuously monitor the average static internal balloon pressure of the balloon fluid 132 within the balloon interior 146.

In a first sensed condition, if the monitored balloon pressure rapidly changes from a high level to zero or near-zero it may indicate the balloon 104 had ruptured. In such situations, the catheter system 100, i.e., the system controller 126, would filter the sensor signal from the pressure sensor assembly 142 to look at a duty cycle or a slowly varying signal. Such a sensor signal indicating a rapid decrease in balloon pressure could be used to automatically lock out the energy source 124 and/or to provide an indicator to the operator to halt the procedure and remove the catheter 102. It is appreciated that during the use of the catheter system 100, if the balloon 104 were to rupture, the procedure would need to be stopped as quickly as possible. With the devices and methods described herein, rupture of the balloon 104 can be quickly and successfully detected, and an indicator or signal can be provided that can be used to halt or rapidly slow the energy source 124. This feature can provide a safety interlock for a potentially hazardous condition in which the balloon fluid 132 may be able to leak out into the patient.

In another sensed condition, the pressure sensor assembly 142 may sense and/or detect relatively small dynamic changes on the larger average static balloon pressure. It is appreciated that these short duration acoustic pulses would be created as plasma-driven bubbles expand and collapse. The catheter system 100 and/or the system controller 126 would filter the sensor signal from the pressure sensor assembly to remove the duty cycle or slowly varying signal to isolate high frequency signals or transient events. Such sensed situation would be correlated with the firing of the plasma-generating energy source 124. If the acoustic transient balloon pressure is detected within some predetermined time interval from the firing of the energy source 124, this would provide an indication that the catheter system 100 functioned correctly, and thus would enable the next event in the sequence.

In yet another sensed condition, if no transient balloon pressure change as sensed by the pressure sensor assembly 142 is detected within a specific time interval after the firing of the energy source 124, this would provide an indication of failure of the catheter system 100, e.g., that the energy source 124 did not fire properly. Such a sensor signal could be used to automatically lock out the energy source 124 and/or to provide an indicator to the operator to halt the procedure and remove the catheter 102.

In still another sensed condition, it is appreciated that the cross-section of the vessel under treatment should change as the procedure progresses. In particular, the vessel wall will relax and expand as calcifications are broken. Such breaking up of the lesions allows the balloon 104 to expand slightly. As such, the internal balloon pressure within the balloon interior 146 of the balloon 104 should decrease incrementally, but it should not rapidly drop to zero. The catheter system 100 and/or the system controller 126 would filter the sensor signal from the pressure sensor assembly 142 to monitor duty cycle and slowly varying signals as with burst detection. Such smaller incremental changes in balloon pressure rather than a rapid drop to zero would provide an indication of acceptable progression of the procedure, and thus provide an indicator of treatment efficacy.

It is understood that the foregoing sensed conditions are not intended to include all such situations that are detectible by the system controller 126, and are not intended to be limiting in any manner. These sensed conditions are provided for ease in understanding and are merely to represent some conditions that would benefit from the devices and/or methods provided herein.

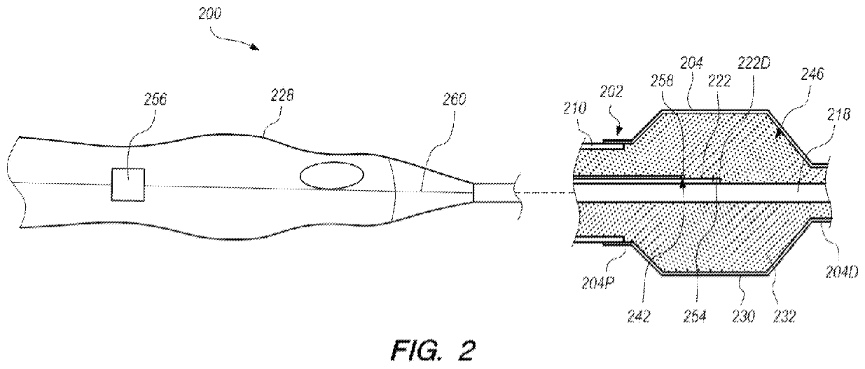

FIG. 2 is a schematic cross-sectional view of a portion of an embodiment of the catheter system 200, including an embodiment of the pressure sensor assembly 242. As described in detail herein, the design of the catheter system 200 can be varied. In various embodiments, as illustrated in FIG. 2, the catheter system 200 can include a catheter 202 including a catheter shaft 210, a balloon 204 having a balloon wall 230 that defines a balloon interior 246, a balloon proximal end 204P, and a balloon distal end 204D, a balloon fluid 232 that is retained substantially within the balloon interior 246, and a guidewire lumen 218 that extends into the balloon interior 246; an energy guide 222 (also sometimes referred to herein as a “light guide”); a handle assembly 228; and the pressure sensor assembly 242. Alternatively, in other embodiments, the catheter system 200 can include more components or fewer components than what is specifically illustrated and described herein. For example, certain components that were illustrated in FIG. 1, e.g., the guidewire 112, the energy source 124 (also sometimes referred to herein as the “light source”), the system controller 126, the GUI 127, the source manifold 136 and the fluid pump 138, are not specifically illustrated in FIG. 2 for purposes of clarity, but would likely be included in any embodiment of the catheter system 200.

The catheter 202, including the catheter shaft 210, the balloon 204, and the guidewire lumen 218, is generally similar in design and operation to what has been described in detail herein above. Thus, such components will not be described in detail again in relation to the embodiment shown in FIG. 2.

As above, the balloon 204 is selectively movable between a collapsed configuration suitable for advancing the catheter 202 through a patient's vasculature, and an expanded configuration suitable for anchoring the catheter 202 in position relative to the treatment site 106 (illustrated in FIG. 1). In some embodiments, the balloon proximal end 204P can be coupled to the catheter shaft 210, and the balloon distal end 204D can be coupled to the guidewire lumen 218. Additionally, the balloon 204 can be inflated with the balloon fluid 232, e.g., from the fluid pump 138 (illustrated in FIG. 1), that is directed into the balloon interior 246 of the balloon 204 via the inflation conduit 140 (illustrated in FIG. 1).

The energy guides 222 can have any suitable design for purposes of generating plasma and/or pressure waves in the balloon fluid 232 within the balloon interior 246. Thus, the particular description of the light guides 222 herein is not intended to be limiting in any manner.

In certain embodiments, the light guides 222 herein can include an optical fiber or flexible light pipe. The light guides 222 herein can be thin and flexible and can allow light signals to be sent with very little loss of strength. The light guides 222 herein can include a core surrounded by a cladding about its circumference. In some embodiments, the core can be a cylindrical core or a partially cylindrical core. The core and cladding of the light guides 222 can be formed from one or more materials, including but not limited to one or more types of glass, silica, or one or more polymers. The light guides 222 may also include a protective coating, such as a polymer. It is appreciated that the index of refraction of the core will be greater than the index of refraction of the cladding.

Each light guide 222 can guide light along its length to a distal portion, i.e., a guide distal end 222D, having at least one optical window (not shown) that is positioned within the balloon interior 246. The light guides 222 can create a light path as a portion of an optical network including the light source 124 (illustrated in FIG. 1). The light path within the optical network allows light to travel from one part of the network to another. Both the optical fiber and the flexible light pipe can provide a light path within the optical networks herein.

The light guides 222 herein can assume many configurations about and/or relative to the catheter shaft 210 of the catheters 202 described herein. In some embodiments, the light guides 222 can run parallel to the longitudinal axis 144 (illustrated in FIG. 1) of the catheter shaft 210 of the catheter 202. In some embodiments, the light guides 222 can be physically coupled to the catheter shaft 210. In other embodiments, the light guides 222 can be disposed along a length of an outer diameter of the catheter shaft 210. In yet other embodiments, the light guides 222 herein can be disposed within one or more light guide lumens within the catheter shaft 210.

The light guides 222 herein can include one or more photoacoustic transducers 254, where each photoacoustic transducer 254 can be in optical communication with the light guide 222 within which it is disposed. In some embodiments, the photoacoustic transducers 254 can be in optical communication with the guide distal end 222D of the light guide 222. Additionally, in such embodiments, the photoacoustic transducers 254 can have a shape that corresponds with and/or conforms to the guide distal end 222D of the light guide 222.

The photoacoustic transducer 254 is configured to convert light energy into an acoustic wave at or near the guide distal end 222D of the light guide 222. It is appreciated that the direction of the acoustic wave can be tailored by changing an angle of the guide distal end 222D of the light guide 222.

It is further appreciated that the photoacoustic transducers 254 disposed at the guide distal end 222D of the light guide 222 herein can assume the same shape as the guide distal end 222D of the light guide 222. For example, in certain non-exclusive embodiments, the photoacoustic transducer 254 and/or the guide distal end 222D can have a conical shape, a convex shape, a concave shape, a bulbous shape, a square shape, a stepped shape, a half-circle shape, an ovoid shape, and the like. It is also appreciated that the light guide 222 can further include additional photoacoustic transducers 254 disposed along one or more side surfaces of the length of the light guide 222.

The light guides 222 described herein can further include one or more diverting features or “diverters” (not shown in FIG. 2) within the light guide 222 that are configured to direct light to exit the light guide 222 toward a side surface, e.g., at or near the guide distal end 222D of the light guide 222, and toward the balloon wall 230. A diverting feature can include any feature of the system herein that diverts light from the light guide 222 away from its axial path toward a side surface of the light guide 222. Additionally, the light guides 222 can each include one or more light windows disposed along the longitudinal or axial surfaces of each light guide 222 and in optical communication with a diverting feature. Stated in another manner, the diverting features herein can be configured to direct light in the light guide 222 toward a side surface, e.g., at or near the guide distal end 222D, where the side surface is in optical communication with a light window. The light windows can include a portion of the light guide 222 that allows light to exit the light guide 222 from within the light guide 222, such as a portion of the light guide 222 lacking a cladding material on or about the light guide 222.

Examples of the diverting features suitable for use herein include a reflecting element, a refracting element, and a fiber diffuser. Additionally, the diverting features suitable for focusing light away from the tip of the light guides 222 herein can include, but are not to be limited to, those having a convex surface, a gradient-index (GRIN) lens, and a mirror focus lens. Upon contact with the diverting feature, the light is diverted within the light guide 222 to the photoacoustic transducer 254 that is in optical communication with a side surface of the light guide 222. As noted, the photoacoustic transducer 254 then converts light energy into an acoustic wave that extends away from the side surface of the light guide 222.

The handle assembly 228 is handled and used by the user or operator to operate, position and control the catheter 202. The design of the handle assembly 228 can vary to suit the design requirements of the catheter system 200. In the embodiment illustrated in FIG. 2, the handle assembly 228 can include circuitry 256 that can form a portion of the system controller 126 (illustrated in FIG. 1). Alternatively, the circuitry 256 can transmit electrical signals such as the sensor signal or sensor output or otherwise provide data to the system controller 126 as described herein. Additionally, or in the alternative, the circuitry 256 can receive electrical signals or data from the pressure sensor assembly 242, e.g., in the form of the sensor signals or sensor output. In one embodiment, the circuitry 256 can include a printed circuit board having one or more integrated circuits, or any other suitable circuitry. In an alternative embodiment, the circuitry 256 can be omitted, or can be included within the system controller 126, which in various embodiments can be positioned outside of the handle assembly 228, e.g., within the system console 123 (illustrated in FIG. 1).

The pressure sensor assembly 242 is configured to sense and/or monitor a balloon pressure within the balloon interior 246 of the balloon 204. As used herein, the “balloon pressure” means the pressure within the balloon interior 246 of the balloon 204 at or substantially contemporaneously with the time the pressure within the balloon interior 246 of the balloon 204 is measured. In the embodiment illustrated in FIG. 2, the pressure sensor assembly 242 can transmit electrical signals to the circuitry 256 within the handle assembly 228, which are then processed and sent to the system controller 126. In an alternative embodiment, the pressure sensor assembly 242 can transmit electrical signals directly to the system controller 126. The design of the pressure sensor assembly 242 can be varied. In the embodiment illustrated in FIG. 2, the pressure sensor assembly 242 includes a pressure sensor 258 and a transmission line 260. Alternatively, the pressure sensor assembly 242 can include more components or fewer components than what is specifically shown and described in relation to FIG. 2.

The pressure sensor 258 can be positioned at any suitable location within the catheter system 200 so that it is in communication with the balloon fluid 232 that is positioned within the balloon interior 246 of the balloon 204. As such, the pressure sensor 258 is configured to provide real-time continuous monitoring of the balloon pressure within the balloon interior 246 in conjunction with electronics or optics in the handle assembly 242 and/or the system controller 126.

In the embodiment illustrated in FIG. 2, and in certain embodiments, the pressure sensor 258 can be positioned substantially within the balloon interior 246 of the balloon 204. With such design, the pressure sensor 258 can directly sense, measure and/or monitor the balloon pressure within the balloon interior 246 of the balloon 204. The pressure sensor 258 can then generate and send a sensor signal or sensor output, e.g., electrical signals regarding the balloon pressure, to the circuitry 256 and/or the system controller 126 via the transmission line 260. As described in greater detail herein, the system controller 126 can then provide appropriate information to the user or operator, e.g., via the GUI 127 (illustrated in FIG. 1), as to the status of operation of the catheter system 200, e.g., success or failure of the firing of the energy source 124, potential rupturing of the balloon 204, and/or efficacy of the treatment process with the catheter system 200, based at least in part on the sensor signal that was received from the pressure sensor 258. Additionally, or in the alternative, in some embodiments, if the sensor signal provides an indication of balloon rupture or failure of the energy source 124 to fire as desired, the system controller 126 can be configured to automatically lock out the energy source 124, and thus stop the normal operation of the catheter system 200. Further, or in the alternative, the system controller 126 can be configured to otherwise process and integrate the sensor signal to determine and/or adjust for proper functioning of the catheter system 200 based at least in part on the sensor signal.

It is appreciated that the specific type of pressure sensor 258 included in the pressure sensor assembly 242 can vary. For example, in one non-exclusive embodiment, the pressure sensor 258 can be an optical fiber sensor that is incorporated directly within the balloon interior 246 of the balloon 204. Such optical fiber sensors use a fiber optic-based or “MEMS” interferometer that is attached to a distal end of an optical fiber. The cavity length of the interferometer is designed to change with local pressure. The proximal end of the optical fiber can be connected to a laser controller. This has optics and a low-power laser that monitors changes in fringe patterns in the interferometer. The change in fringe count is correlated directly to pressure. Alternatively, another suitable type of pressure sensor 258 can be used.

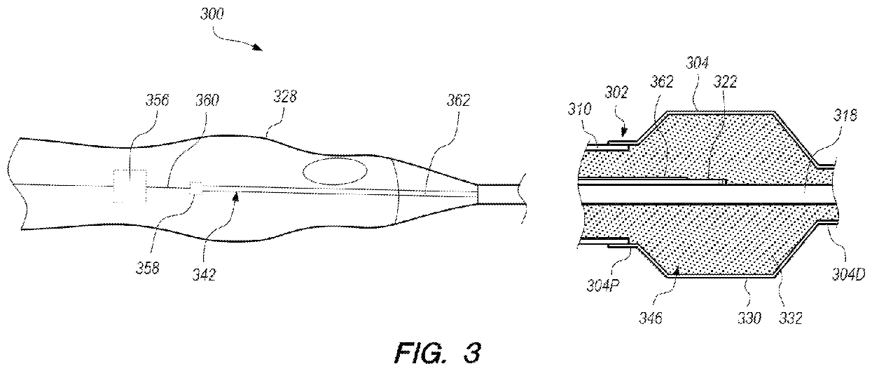

FIG. 3 is a schematic cross-sectional view of a portion of an embodiment of the catheter system 300, including another embodiment of the pressure sensor assembly 342. The design of the catheter system 300 is somewhat similar to the embodiments illustrated and described herein above. In particular, in the embodiment shown in FIG. 3, the catheter system 300 can again include a catheter 302 including a catheter shaft 310, a balloon 304 having a balloon wall 330 that defines a balloon interior 346, a balloon proximal end 304P, and a balloon distal end 304D, a balloon fluid 332 that is retained substantially within the balloon interior 346, and a guidewire lumen 318 that extends into the balloon interior 346; an energy guide 322 (also sometimes referred to herein as a “light guide”); a handle assembly 328; and the pressure sensor assembly 342. Alternatively, in other embodiments, the catheter system 300 can include more components or fewer components than what is specifically illustrated and described herein. For example, certain components that were illustrated in FIG. 1, e.g., the guidewire 112, the energy source 124 (also sometimes referred to herein as the “light source”), the system controller 126, the GUI 127, the source manifold 136 and the fluid pump 138, are not specifically illustrated in FIG. 3 for purposes of clarity, but would likely be included in any embodiment of the catheter system 300.

The catheter 302, including the catheter shaft 310, the balloon 304, and the guidewire lumen 318, is generally similar in design and operation to what has been described in detail herein above. Thus, such components will not be described in detail again in relation to the embodiment shown in FIG. 3. Additionally, the light guide 322 is also generally similar in design and function to what has been described in detail herein above. Thus, the light guide 322 will also not be described in detail again in relation to the embodiment shown in FIG. 3.

The balloon 304 is selectively movable between a collapsed configuration suitable for advancing the catheter 302 through a patient's vasculature, and an expanded configuration suitable for anchoring the catheter 302 in position relative to the treatment site 106 (illustrated in FIG. 1). In some embodiments, the balloon proximal end 304P can again be coupled to the catheter shaft 310, and the balloon distal end 304D can again be coupled to the guidewire lumen 318. Additionally, the balloon 304 can be inflated with the balloon fluid 332, e.g., from the fluid pump 138 (illustrated in FIG. 1), that is directed into the balloon interior 346 of the balloon 304 via the inflation conduit 140 (illustrated in FIG. 1).

The handle assembly 328 is handled and used by the user or operator to operate, position and control the catheter 302. The design of the handle assembly 328 can vary to suit the design requirements of the catheter system 300. In the embodiment illustrated in FIG. 3, the handle assembly 328 can include circuitry 356 that can form a portion of the system controller 126 (illustrated in FIG. 1). In this embodiment, the circuitry 356 can be substantially similar in design and function as described in the previous embodiment. In an alternative embodiment, the circuitry 356 can be omitted, or can be included within the system controller 126, which in various embodiments can be positioned outside of the handle assembly 328, e.g., within the system console 123 (illustrated in FIG. 1).

The pressure sensor assembly 342 is again configured to sense and/or monitor a balloon pressure within the balloon interior 346 of the balloon 304. As with the previous embodiment, the pressure sensor assembly 342 can transmit electrical signals, e.g., sensor signals or sensor output, to the circuitry 356 within the handle assembly 328, which are then processed and sent to the system controller 126. Alternatively, the pressure sensor assembly 342 can transmit electrical signals directly to the system controller 126.

The design of the pressure sensor assembly 342 can be varied. In the embodiment illustrated in FIG. 3, the pressure sensor assembly 342 includes a pressure sensor 358, a transmission line 360, and a tubular member 362 that defines a sensor lumen (an interior of the tubular member 362). Alternatively, the pressure sensor assembly 342 can include more components or fewer components than what is specifically shown and described in relation to FIG. 3. For example, in one non-exclusive alternative embodiment, the pressure sensor assembly 342 can be designed without the tubular member 362.

The pressure sensor 358 can be positioned at any suitable location within the catheter system 300 so that it is in communication with the balloon fluid 332 that is positioned within the balloon interior 346 of the balloon 304. As such, the pressure sensor 358 is configured to provide real-time continuous monitoring of the balloon pressure within the balloon interior 346 in conjunction with electronics or optics in the handle assembly 328 and the system controller 126.

In certain embodiments, the pressure sensor 358 is positioned outside of the balloon interior 346 of the balloon 304. In such embodiments, the pressure sensor 358 can be located inside the fluidic channel of the catheter system 300, which includes the handle assembly 328, endoflator, irrigation tubes or catheter shaft 310 of the catheter 302. Since the balloon fluid 332 in the balloon interior 346 is incompressible, and in communication with the handle assembly 328 through the catheter shaft 310, the static fluid pressure in the catheter shaft 310, the balloon interior 346 and the handle assembly 328 are equal.

In the embodiment illustrated in FIG. 3, the pressure sensor 358 is positioned within the handle assembly 328. Alternatively, the pressure sensor 358 can be positioned anywhere between the balloon 304 and the handle assembly 328. Still, alternatively, the pressure sensor 358 can be positioned between the handle assembly 328 and the system controller 126.

In various embodiments, the pressure sensor 358 is in fluid communication with the balloon fluid 332 within the balloon interior 346 of the balloon 304. In the embodiment illustrated in FIG. 3, the tubular member 362 extends from the pressure sensor 358 to the balloon interior 346. Thus, the pressure sensor 358 is in fluid communication with the balloon interior 346 via the tubular member 362. The tubular member 362 can be a relatively small diameter tube that can transmit the balloon pressure within the balloon interior 346 directly to the pressure sensor 358. Alternatively, in embodiments that do not include the tubular member 362, the pressure sensor 358 can be in fluid communication with the balloon interior 346 via the catheter shaft 310.

The pressure sensor 258 can then generate and send a sensor signal or sensor output, e.g., electrical signals regarding the balloon pressure, to the circuitry 256 and/or the system controller 126 via the transmission line 260. As described herein, the system controller 126 can then provide appropriate information to the user or operator, e.g., via the GUI 127 (illustrated in FIG. 1), as to the status of operation of the catheter system 300, e.g., success or failure of the firing of the energy source 124, potential rupturing of the balloon 304, and/or efficacy of the treatment process with the catheter system 300, based at least in part on the sensor signal that was received from the pressure sensor 358. Additionally, or in the alternative, in some embodiments, if the sensor signal provides an indication of balloon rupture or failure of the energy source 124 to fire as desired, the system controller 126 can be configured to automatically lock out the energy source 124, and thus stop the normal operation of the catheter system 300. Further, or in the alternative, the system controller 126 can be configured to otherwise process and integrate the sensor signal to determine and/or adjust for proper functioning of the catheter system 300 based at least in part on the sensor signal.

The specific type of pressure sensor 358 included in the pressure sensor assembly 342 can vary. For example, in one non-exclusive embodiment, the pressure sensor 358 can be a conventional diaphragm or “MEMS” sensor that can be located inside the fluidic channel from the handle assembly 328 to the balloon interior 346. As noted, the incompressibility of the balloon fluid 332 results in the static fluid pressure in the catheter shaft 310, the balloon interior 346 and the handle assembly 328 being equal. Thus, in some embodiments, the pressure sensor 358 can directly monitor and/or sense the fluid pressure at its location, which will be deemed to be substantially equal to the balloon pressure within the balloon interior 346. As such, the pressure sensor assembly 342 can be designed without the tubular member 362 even when the pressure sensor 358 is positioned outside the balloon interior 346. Additionally, the viscosity of the balloon fluid 332 moving through the narrow cross-sections produces a time delay in transfer of short-duration or transient events such as pulses from the pressure wave event. Alternatively, an optical fiber sensor, a diaphragm sensor, or any other suitable type of pressure sensor 358 can be used within the pressure sensor assembly 342.

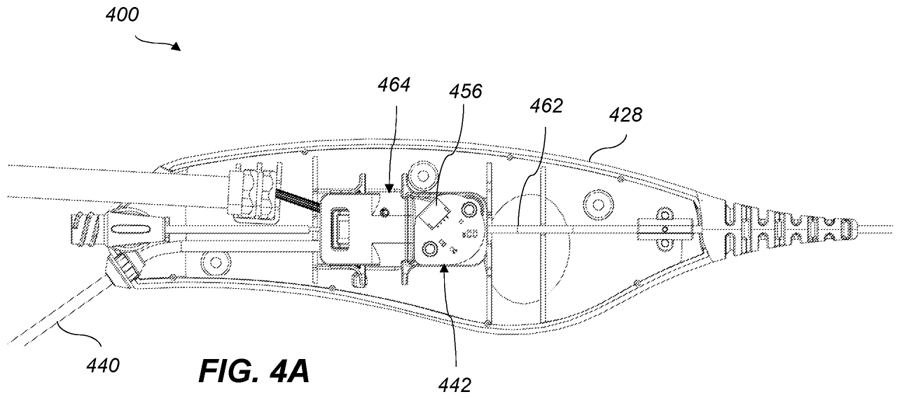

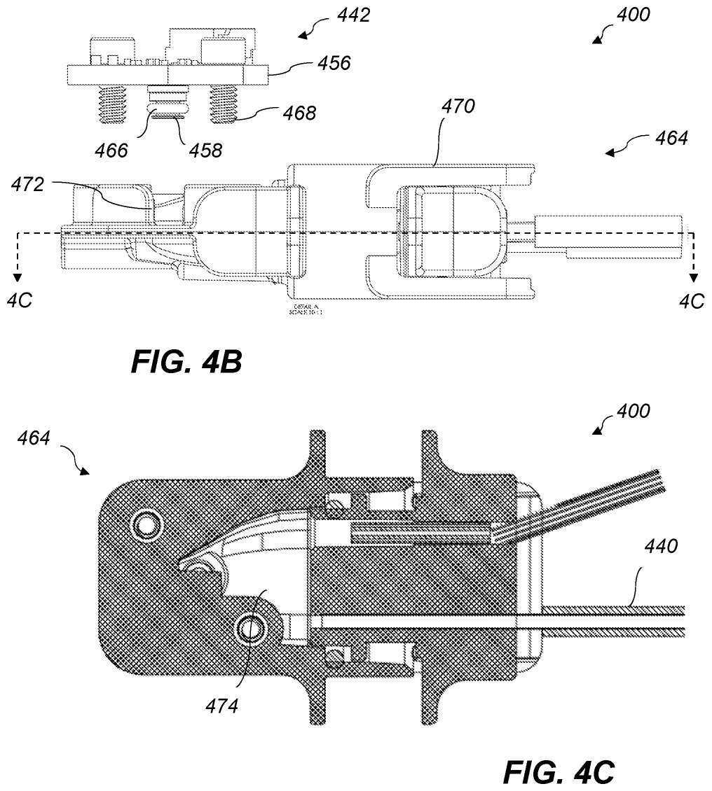

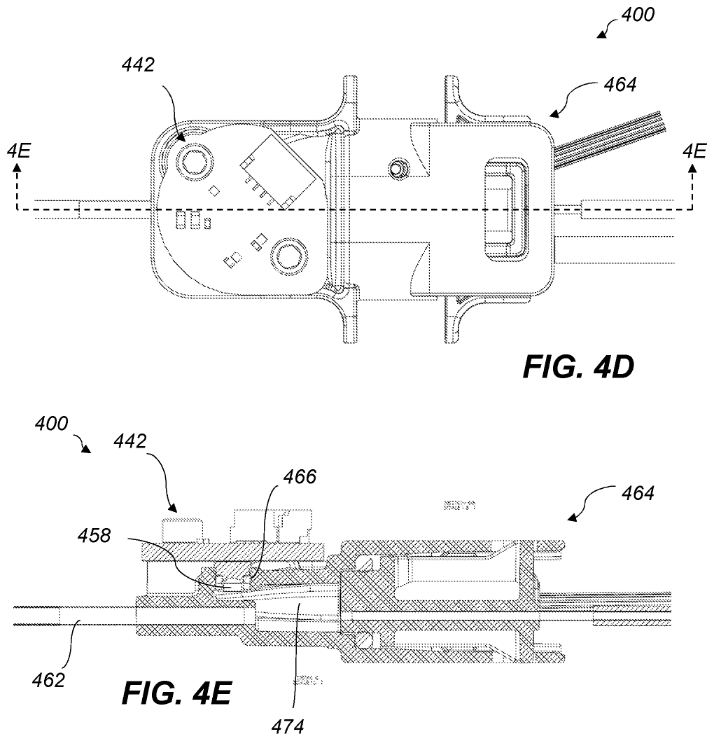

FIG. 4A is a schematic cross-sectional view of a portion of an embodiment of the catheter system 400, including another embodiment of the pressure sensor assembly 442. The design of the catheter system 400 is somewhat similar to the embodiments illustrated and described herein above. In particular, in the embodiment shown in FIGS. 4A-4F, the catheter system 400 can again include a handle assembly 428, an inflation conduit 440, a pressure sensor assembly 442, circuitry 456 (illustrated in FIG. 4B), a pressure sensor 458 (illustrated in FIG. 4B), and a tubular member 462. Alternatively, in other embodiments, the catheter system 400 can include more components or fewer components than what is specifically illustrated and described herein. For example, certain components that were illustrated in FIG. 1, e.g., the guidewire 112, the energy source 124 (also sometimes referred to herein as the “light source”), the system controller 126, the GUI 127, the source manifold 136 and the fluid pump 138, are not specifically illustrated in FIGS. 4A-4F for purposes of clarity, but would likely be included in any embodiment of the catheter system 400.

The handle assembly 428, the inflation conduit 440, the pressure sensor assembly 442, the circuitry 456, the pressure sensor 458, and the tubular member 462 are generally similar in design and operation to what has been described in detail herein above. Thus, such components will not be described in detail again in relation to the embodiment shown in FIGS. 4A-4F except as otherwise indicated.

The handle assembly 428 is handled and used by the user or operator to operate, position and control the catheter 102 (e.g., as illustrated in FIG. 1). The design of the handle assembly 428 can vary to suit the design requirements of the catheter system 300. In the embodiment illustrated in FIGS. 4A-4F, the handle assembly 428 can include the pressure sensor assembly 442 that includes circuitry 456 that can form a portion of the system controller 126 (illustrated in FIG. 1) and a manifold assembly.

In this embodiment, the circuitry 456 can be substantially similar in design and function as described in the previous embodiment. In an alternative embodiment, the circuitry 456 can be omitted, or can be included within the system controller 126, which in various embodiments can be positioned outside of the handle assembly 428, e.g., within the system console 123 (illustrated in FIG. 1). The circuitry 456 can include a printed circuit board, such as shown in FIG. 4A-4F.

The manifold assembly 464 is positioned within the handle assembly 428 so that the pressure sensor assembly 442 can sense and/or monitor a balloon pressure within the balloon interior 346 (illustrated in FIG. 3) of the balloon 304 (illustrated in FIG. 3). The measurement of the pressure can be taken at the manifold assembly 464. The manifold assembly 428 can be in fluid communication with (i) the balloon fluid 332 within the balloon interior 346, (ii) the pressure sensor assembly 442, and (iii) the inflation conduit 440.

The design of the manifold assembly 464 can be varied. The manifold assembly 464 can be in fluid communication between the inflation conduit 440 and the tubular member 462 so that inflation flows from the inflation conduit 440 through the manifold assembly 464 through the tubular member 462 into the balloon 304. In some embodiments, the balloon fluid 332 is configured to flow first through the inflation conduit 440 and subsequently through the manifold assembly 464 before entering the balloon interior 346. Inflation media, such as the balloon fluid 332, can be pushed through this inflation pathway from the inflation conduit 440 to the balloon 304 so that the balloon 304 can be inflated. The pressure sensor assembly 442 can be selectively coupled to the manifold assembly 464.

FIG. 4B is a partially exploded side view of a portion of an embodiment of the catheter system 400, including another embodiment of the pressure sensor assembly 442 and a manifold assembly 464.

The pressure sensor assembly 442 can be in fluid communication with the balloon interior 346 (illustrated in FIG. 3) and the inflation conduit 440. The design of the pressure sensor assembly 442 can be varied. In the embodiment illustrated in FIGS. 4A-4F, the pressure sensor assembly 442 includes circuitry 456, a pressure sensor 458, a pressure sensor sealer 466, and a pressure sensor fastener 468. Alternatively, the pressure sensor assembly 442 can include more components or fewer components than what is specifically shown and described in relation to FIG. 4B.

The pressure sensor sealer 466 forms a fluid-tight seal between the pressure sensor 458 and the manifold assembly 464. The pressure sensor sealer 466 inhibits and/or reduces the likelihood of changes in pressure within the balloon 304 and the manifold assembly 464. The design of the pressure sensor sealer 466 can be varied. In some embodiments, the pressure sensor sealer 466 can include an o-ring that encircles the pressure sensor 458.

The pressure sensor fastener 468 can fasten the pressure sensor assembly 442 to the manifold assembly 464. The design of the pressure sensor fastener 468 can be varied. In some embodiments, the pressure sensor fastener 468 is threaded. While only one pressure sensor fastener 468 is identified in FIG. 4B, it is appreciated that the pressure sensor assembly 442 can include any suitable number of pressure sensor fasteners 468.

The manifold assembly 464 can include a manifold body 470 having a manifold body aperture 472. The manifold body 470 can be coupled to the handle assembly 428, the inflation conduit 440, the pressure sensor assembly 442, and/or the tubular member 462. The manifold body aperture 472 can be configured to receive the pressure sensor 458 and the pressure sensor sealer 466. The pressure sensor sealer 466 can form a seal between the pressure sensor 458 and the manifold body 470 when the pressure sensor 458 and the pressure sensor sealer 466 are positioned within the manifold body aperture 472.

FIG. 4C is a cross-sectional top view of a portion of the embodiment of the catheter system 400, including the manifold assembly 464 taken along lines 4C-4C in FIG. 4B. The manifold assembly 464 can include a manifold retainer 474 that is configured to receive and retain the balloon fluid 332 and/or any suitable inflation media. The manifold retainer 474 can be positioned within the manifold body 470. The pressure sensor 458 can be positioned so that it contacts the manifold retainer 474 so that the pressure sensor 458 can sense the balloon pressure of the balloon fluid 332 based on the balloon fluid 332 retained within the manifold retainer 474. As shown in the cross-sectional top view illustrated in FIG. 4C, the manifold retainer 474 has a substantially triangular cross-sectional shape when viewed from the cross-sectional top view.

FIG. 4D is a top view of a portion of the embodiment of the catheter system 400 illustrated in FIGS. 4A-4C. As shown from the top view, the pressure sensor assembly 442 can have a substantially circular perimeter and the manifold assembly 464 can have a substantially rectangular perimeter. However, it is appreciated that the pressure sensor assembly 442 and the manifold assembly 464 can have any suitable perimeter and/or shape.

FIG. 4E is a cross-sectional side view of a portion of the embodiment of the catheter system 400 taken along line 4E-4E in FIG. 4D. As shown in FIG. 4E, the manifold retainer 474 has a substantially rectangular cross-sectional shape when viewed from the cross-sectional side view.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The foregoing discussion of the disclosure has been presented for purposes of illustration and description. The foregoing is not intended to limit the disclosure to the form or forms disclosed herein. In the foregoing description, for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It is, therefore, intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A catheter system for disrupting calcification at a treatment site within or adjacent to a vessel wall or heart valve, the catheter system comprising:
an energy source that generates energy;
a balloon that is positionable substantially adjacent to the treatment site, the balloon having a balloon wall that defines a balloon interior, the balloon interior being configured to receive a balloon fluid;
an energy guide that is configured to receive energy from the energy source, the energy guide guiding the energy into the balloon interior;
an inflation conduit that is in fluid communication with the balloon interior, the inflation conduit being configured to convey the balloon fluid into the balloon interior; and
a pressure sensor assembly that is configured to sense a balloon pressure of the balloon fluid within the balloon interior, the pressure sensor assembly being in fluid communication with the balloon interior and the inflation conduit.

2. The catheter system of claim 1 further comprising a handle assembly that is coupled to the balloon, the handle assembly being positioned spaced apart from the balloon, the handle assembly being usable by a user to operate the catheter system, the pressure sensor assembly being positioned within the handle assembly.

3. The catheter system of claim 2 wherein further comprising a manifold assembly that is coupled to the handle assembly, the manifold assembly being in fluid communication with (i) the balloon fluid within the balloon interior, (ii) the pressure sensor assembly, and (iii) the inflation conduit.

4. The catheter system of claim 3 wherein the inflation fluid is configured to flow first through the inflation conduit and subsequently through the manifold assembly before entering the balloon interior.

5. The catheter system of claim 3 wherein the manifold assembly includes a manifold retainer that is configured to receive and retain the balloon fluid.

6. The catheter system of claim 5 wherein the pressure sensor assembly includes a pressure sensor that contacts the manifold retainer so that the pressure sensor can sense the balloon pressure of the balloon fluid based on the balloon fluid retained within the manifold retainer.

7. The catheter system of claim 3 wherein the pressure sensor assembly includes a pressure sensor, the pressure sensor including a printed circuit board.

8. The catheter system of claim 7 wherein the manifold assembly includes a manifold body, the pressure sensory assembly being coupled to the manifold body so that the pressure sensor extends within the manifold body.

9. The catheter system of claim 8 wherein the pressure sensor assembly includes a pressure sensor sealer that forms a seal between the pressure sensor and the manifold body.

10. The catheter system of claim 8 wherein the manifold body includes a pressure sensor aperture that is configured to receive the pressure sensor.

11. A catheter system for disrupting calcification at a treatment site within or adjacent to a vessel wall or heart valve, the catheter system comprising:
a balloon that is positionable substantially adjacent to the treatment site, the balloon having a balloon wall that defines a balloon interior, the balloon interior being configured to receive a balloon fluid;
an inflation conduit that is in fluid communication with the balloon interior, the inflation conduit being configured to convey the balloon fluid into the balloon interior; and
a pressure sensor assembly that is configured to sense a balloon pressure of the balloon fluid within the balloon interior, the pressure sensor assembly being in fluid communication with the balloon interior and the inflation conduit.

12. The catheter system of claim 11 further comprising a handle assembly that is coupled to the balloon, the handle assembly being positioned spaced apart from the balloon, the handle assembly being usable by a user to operate the catheter system, the pressure sensor assembly being positioned within the handle assembly.

13. The catheter system of claim 12 further comprising a manifold assembly that is coupled to the handle assembly, the manifold assembly being in fluid communication with (i) the balloon fluid within the balloon interior, (ii) the pressure sensor assembly, and (iii) the inflation conduit.

14. The catheter system of claim 13 wherein the inflation fluid is configured to flow first through the inflation conduit and subsequently through the manifold assembly before entering the balloon interior.

15. The catheter system of claim 13 wherein the manifold assembly includes a manifold retainer that is configured to receive and retain the balloon fluid.

16. The catheter system of claim 15 wherein the pressure sensor assembly includes a pressure sensor that contacts the manifold retainer so that it can sense the balloon pressure of the balloon fluid based on the balloon fluid retained within the manifold retainer.

17. The catheter system of claim 13 wherein the pressure sensor assembly includes a pressure sensor, the pressure sensor including a printed circuit board.

18. The catheter system of claim 17 wherein the manifold assembly includes a manifold body, the pressure sensory assembly being coupled to the manifold body so that the pressure sensor extends within the manifold body.

19. The catheter system of claim 18 wherein the pressure sensor assembly includes a pressure sensor sealer that forms a seal between the pressure sensor and the manifold body.

20. A catheter system for disrupting calcification at a treatment site within or adjacent to a vessel wall or heart valve, the catheter system comprising:
an energy source that generates energy;
a balloon that is positionable substantially adjacent to the treatment site, the balloon having a balloon wall that defines a balloon interior, the balloon interior being configured to receive a balloon fluid;
an energy guide that is configured to receive energy from the energy source, the energy guide guiding the energy into the balloon interior;
a handle assembly that is coupled to the balloon, the handle assembly being positioned spaced apart from the balloon, the handle assembly being usable by a user to operate the catheter system;
an inflation conduit that is in fluid communication with the balloon interior, the inflation conduit being coupled to the handle assembly, the inflation conduit being configured to convey the balloon fluid into the balloon interior; and
a pressure sensor assembly that is configured to sense a balloon pressure of the balloon fluid within the balloon interior, the pressure sensor assembly being in fluid communication with the balloon interior and the inflation conduit, the pressure sensor assembly being positioned within the handle assembly.
